(19) United States
(12) Patent Application Publication
(10) Pub. No.: US 2015/0141904 A1
(43) Pub. Date: May 21, 2015

(54) COMPRESSED GAS MOTOR FOR OPERATION OF A LAVAGE SYSTEM

(71) Applicant: Heraeus Medical GmbH, Wehrheim (DE)

(72) Inventor: Sebastian Vogt, Erfurt (DE)

(21) Appl. No.: 14/524,765

(22) Filed: Oct. 27, 2014

(30) Foreign Application Priority Data
Nov. 18, 2013 (DE) 10 2013 223 501.1

(51) Int. Cl. A61M 11/02; F01B 11/00; A61M 3/02

(52) U.S. Cl. CPC A61M 11/02; A61M 3/0254; F01B 11/007
USPC 604/24; 91/403; 91/471

(57) ABSTRACT

A compressed gas motor and methods comprise a working plunger and a control plunger arranged in the internal space between the working plunger and a closed rear side such as to be mobile in linear direction. The control plunger, in a first position, covers a gas outlet opening and does not cover the gas inlet opening and, in a second position, covers the gas inlet opening and does not cover the gas outlet opening. The control plunger is supported as in a bearing such as to be mobile with respect to the working plunger, and a catch element is arranged on said working plunger and/or control plunger and pulls the control plunger in the direction of the working plunger, at a first distance, when the working plunger moves away from the control plunger, as well as a lavage system comprising said compressed gas motor.

The invention relates to a compressed gas motor comprising a working plunger, an internal space that is closed on a rear side and has the working plunger arranged in it such that the working plunger is mobile in linear direction, a restoring element that exerts a force on the working plunger, at least part of the time, in the direction of the rear side, a gas inlet opening for supplying a compressed gas into the internal space and a gas outlet opening for discharging the gas from the internal space. The invention also relates to a lavage system comprising a compressed gas motor of this type.

The invention also relates to the use of a compressed gas motor of this type and to a method for producing a periodical motion by means of a compressed gas.

Accordingly, the object of the invention is a simplified compressed gas motor that can essentially be made from inexpensive plastic materials and is intended to drive devices that are operated only once, for a short period of time, in particular medical spraying devices for single use made of plastic materials. Moreover, a pump for liquids that is driven by the compressed gas motor is described. Moreover, the use of the compressed gas motor for driving a pump for dispensing a lavage fluid of a medical spraying device (i.e. a lavage system) intended for single use only is proposed.

Medical rinsing systems are used widely in surgery to clean tissue areas. Said rinsing systems are called lavage systems. The lavage systems and rinsing liquids are used to produce spray jets that impinge on the tissue areas to be cleaned and exert a mechanical cleaning effect on said tissue areas. Specifically during the implantation of articular endoprostheses and during septic revisions, lavage systems have essential significance (R. M. Sherman et al.: The role of lavage in preventing hemodynamic and blood-gas changes during cemented arthroplasty. J. Bone Joint. Surg. 1983; 65-A: 500-506; S. J. Breusch et al.: Zementierte Hüftendoprothetik: Verminderung des Fettembolierisikos in der zementierten Hüftendoprothetik mittels gepulster Druckspülung. Orthopädie 2000; 29: 578-586; S. J. Breusch et al.: Lavage technique in THA: Jet-lavage Produces Better Cement Penetration Than Syringe-Lavage in the Proximal Femur. J. Arthroplasty. 200; 15(7): 921-927; R. J. Byrick et al.: High-volume, high pressure pulsatile lavage during cemented arthroplasty. J. Bone Joint Surg. 1989; 81-A: 1331-1336; J. Christie et al.: Medullary lavage reduces embolic phenomena and cardiopulmonary changes during cemented hemiarthroplasty. J. Bone Joint Surg. 1995; 77-B: 456-459).

Pulsed lavage systems have been known for a long time, for example from U.S. Pat. No. 4,583,531 A, U.S. Pat. No. 4,278,078 A, and U.S. Pat. No. 5,542,918 A. The lavage systems currently on the market are driven by means of electrical motors (for example InterPulse® Jet lavage made by Stryker GmbH & Co. KG) or compressed air (for example PALAVAGE® made by Heraeus Medical GmbH). Hand-held, electrically-driven lavage systems have also proven useful. However, a large battery block or rechargeable battery block, which only has a limited charge capacity due to its nature, always needs to be taken along. Battery blocks and rechargeable battery blocks are viewed critically in terms of their environmental impact. Compressed gas-driven lavage systems are advantageous in that compressed air is usually available in the operating theatre in unlimited quantities and thus allows rinsing liquid to be sprayed for any desired time without the energy supply being limited.

Systems driven by compressed air or other compressed gases usually utilise a compressed gas motor as the drive. Most compressed gas motors for lavage systems are lamellar compressed gas motors. The compressed gas motor generates a rotary motion which is then translated into an oscillating linear motion. The oscillating linear motion is utilised to convey momentum to small volumes of a rinsing medium. It is common in this context to arrange at least one membrane between the drive and the inlet of rinsing liquid in order to be able to transmit the pulses to the rinsing liquid. This generates spray puffs at high pulse rates of 2,000 to 3,000 pulses per minute. This means that the compressed gas motor needs to be manufactured at high precision in order to tolerate such high rotation rates. Moreover, sufficiently stable storage must be available. For these reasons, the compressed gas motor is the most expensive component of common compressed air-driven lavage systems. Therefore, the compressed gas motor is generally arranged in a handle made of metal or other durable materials such that this component can be used multiply after appropriate reprocessing and sterilisation.

One fundamental shortcoming of lamellar compressed gas motors is that very large gas volumes are needed to operate them. The efficiency of lamellar compressed gas motors is relatively low. However, it would be desirable to have an inexpensive compressed gas motor that can be operated with smaller gas volumes such that lavage systems can be driven independent of location, i.e. without external energy source, and independent of a stationary compressed air supply through the use of small-volume gas cartridges, preferably gas cartridges containing liquefied gas. The use of an inexpensive compressed gas motor made from inexpensive plastic materials would allow compressed gas-driven lavage system to be produced that are intended for single use only and can be disposed inexpensively after use. This is particularly advantageous in medical surgery for hygiene reasons as well.

Compressed gas motors containing plungers have been known in general for a long time. For operation of said compressed gas motors, the inflow of compressed gas must be controlled by inlet valves and the outflow of the relaxed gas after expansion of the gas must proceed through outlet valves. This means that gas inlet valve and gas outlet valve must be coordinated accurately in time and are opened and closed depending on the plunger position. Accordingly, precise time control of the valve is essential for function in this context. The valve control can apply one of several principles in this context. Frequently, the valve control involves cams arranged on a camshaft. One persistent issue of compressed gas motors is that a so-called “dead centre” needs to be overcome at which the compressed gas motor might come to a standstill without the compressed gas being able to start it again. This is often attained by means of suitable rotating flywheel masses. The compressed gas motors utilising plungers that are thus far common according to the prior art are very complex in structure and necessitate exact time control of the valve. Due to their complexity and high cost, said compressed gas motors are not well-suited for lavage systems for single use.

A compressed gas motor of this type is known from WO 2012/038003 A1. The compressed gas motor described in this reference has a two-part plunger with an intervening space and a passage through one of the plungers. This makes the structure of the motor particularly easy and inexpensive.

But there is always a desire to have a compressed gas motor with a less expensive structure. Moreover, there is also a need to provide a compressed gas motor that can be operated at a higher frequency and/or with a larger force.

However, compressed gas motors of this type can be used not only for lavage systems, but also in all applications, in which a compressed gas, and in particular compressed air, is available and an inexpensive drive is advantageous. Said requirements are evident, for example, in shaker facilities, in which bulk goods or powder need(s) to be transported, filled into containers and/or dosed. Likewise, said compressed gas motors can be used advantageously as pumps providing lubricants. Moreover, the use in toys that can be manufactured inexpensively would also be advantageous.

Accordingly, it is the object of the invention to overcome the disadvantages of the prior art. Specifically, the object is to discover an inexpensive and reliable compressed gas motor that can be used for the afore-mentioned purposes. It is an object of the invention to develop a maximally simplified plunger-equipped compressed gas motor that can generate a periodical linear plunger motion. It is important in this context that the compressed gas motor works without needing complex costly valve systems and is simplified to the extent that the components of the compressed gas motor can be produced inexpensively through injection moulding of plastic materials. Valve systems that take up a large volume and need to be positioned separately from the compressed gas motor need to be avoided. Accordingly, the requisite valve functions shall be integrated into the compressed gas motor in space-saving manner in order to enable the use of the compressed gas motor as a drive in hand-pieces of lavage systems. Ideally, the time control of the valve functions should be implemented appropriately such that there is no “dead centre” at any point of the plunger motion. Moreover, a simplified pump for integration into hand-held lavage systems is to be developed along with the compressed gas motor. A pump driven by the compressed gas motor shall be simplified to the extent that it can be produced inexpensively enough to allow it to be used in lavage systems intended for single use only.

The objects of the invention are met by a compressed gas motor comprising a working plunger, an internal space that is closed on a rear side and has the working plunger arranged in it such that the working plunger is mobile in linear direction, a restoring element that exerts a force on the working plunger, at least part of the time, in the direction of the rear side, a gas inlet opening for supplying a compressed gas into the internal space, and a gas outlet opening for discharging the gas from the internal space, that is characterised in that a control plunger is arranged in the internal space between the working plunger and the closed rear side such as to be mobile in linear direction, whereby the control plunger, in a first position, covers the gas outlet opening and does not cover the gas inlet opening and, in a second position, covers the gas inlet opening and does not cover the gas outlet opening, the control plunger is supported as in a bearing such as to be mobile with respect to the working plunger, and a catch element is arranged on said working plunger and/or control plunger and pulls the control plunger in the direction of the working plunger, at a first distance, when the working plunger moves away from the control plunger.

In a compressed gas motor that is appropriately set-up in vertical direction, it is even feasible to forego a physical restoring element, since the working plunger can be restored by gravity in this case. Accordingly, the invention can provide the restoring element to be formed by setting-up the compressed gas motor vertically by means of which the working plunger is moved forward towards the rear side by means of gravity. The compressed gas motor then has a particularly simple structure. In order to be able to position the compressed gas motor variably with respect to the surface of the earth, as may be required in hand-held lavage systems, the invention can preferably provide the restoring element to be a physical restoring element, in particular an elastic spring, such as, for example, a steel spring and/or a gas-operated spring system. The elastic spring can either be a tension spring or a compression spring. As a compression spring, the elastic spring is arranged in the internal space between the working plunger and one front end of the internal space opposite from the rear side. As a tension spring, the elastic spring is arranged in the internal space between the rear side of the internal space and the working plunger. For this purpose, the tension spring needs to be anchored both on the rear side of the internal space and on the working plunger. It is preferred in this context that the tension spring is anchored on the catch element, whereby the catch element is firmly connected to the working plunger. Particularly preferably, the catch element is connected to the working plunger by means of a rod or a catch pin. Even more particularly preferably, the catch pin, or the rod as the case may be, and the working plunger are provided to be the same part. Alternatively or in addition, a gas-operated spring can just as well be used as restoring element, for example in that the internal space between the working plunger and the front end of the internal space is closed with respect to the exterior in gas-tight and pressure-tight manner.

The invention proposes the internal space, at least regions thereof, to be cylindrical or to be cylindrical in the region of a working space of the working plunger or in the entire swept volume of the working plunger and control plunger.

The cylindrical internal space need not be completely cylindrical. It is sufficient that the swept volume of the working plunger is cylindrical. Preferably, the swept volume of the control plunger is cylindrical as well. The swept volume of the control cylinder does not have to be cylindrical if cut-outs or other recesses are provided therein that serve to allow the compressed gas to get past the control plunger into the space between the control plunger and the rear side of the internal space. The space between the maximal travel of the working cylinder in the direction of the front end of the internal space and the front end of the internal space, in which the restoring element is preferably arranged (particularly preferably provided as an elastic spring), does not have to be cylindrical. However, the structure of the compressed gas motor is simplified if this part of the internal space is cylindrical as well. A cylinder in the scope of the invention and according to general definition is a body bounded by two parallel, planar, congruent surfaces (base surface and cover surface) and a jacket surface and/or cylinder surface, whereby the jacket surface is formed by parallel straight lines. This means that the cylinder is generated through shifting a planar surface or curve along a straight line that is not positioned in said plane. The height of the cylinder is given by the distance between the two planes, in which base surface and cover surface are situated.

If the straight lines are perpendicular to base surface and cover surface, the structure is called a straight cylinder. The straight cylindrical geometry of the internal space is preferred according to the invention, but usually relates only to a partial region of the entire internal space. A straight circular cylinder in the scope of the present invention is therefore only a special case of a cylindrical geometry.

The working plunger closes tightly against the internal walls in all positions such that the internal space is always separated into two separate chambers by the working plunger. Preferably, said separation is gas-tight and pressure-tight.

The invention can provide the length of the control plunger in axial direction of the internal space to be at least equal to the sum of the axial distance of the gas inlet opening from the gas outlet opening and the axial cross-sections of the gas inlet opening and gas outlet opening. The axial cross-sections of the gas inlet opening and gas outlet opening are the cross-sections of the gas inlet opening and gas outlet opening in axial direction of the internal space, which, in particular, is cylindrical.

Preferably, the linear motion of the working plunger and control plunger proceeds along a straight line that corresponds to the axis of symmetry of the cylindrical internal space.

In a compressed gas motor according to the invention, the invention can provide the control plunger, in a third position between the first position and the second position, to cover both the gas inlet opening and the gas outlet opening, in particular to cover them fully.

This ensures that the compressed gas motor does not stop in an intermediate position, in which the gas inlet opening and the gas outlet opening are open at the same time, and stays arrested due to this “short-circuiting”. The inertia of the working plunger and/or the force of the restoring element make sure that these two openings do not stay closed permanently.

Moreover, the invention can provide at least one spacer to be arranged on the side of the control plunger facing the working plunger and/or at least one spacer to be arranged on the side of the working plunger facing the control plunger, such that an intervening space arises between the working plunger and the control plunger when these touch against each other, and the gas inlet opening, in the first position, to exit into said intervening space, whereby said at least one spacer adjusts a second distance between the working plunger and the control plunger when working plunger and control plunger touch against each other, whereby the second distance is smaller than the first distance that is being adjusted by the catch element.

The spacer can be formed by means of a tube along the central axis of the internal space through which the catch and/or the catch pin or the rod extends on which the catch is fastened to the working plunger or to the control plunger. Alternatively, the at least one spacer can just as well be provided as a pin. Preferably, the spacer or spacers and the working plunger or control plunger are provided to be the same part. Having the spacer or spacers ensures that the compressed gas motor can be made to run in any case by supplying the compressed gas between the working plunger and the control plunger.

It is essential to the invention that the distance between the working plunger and the control plunger enlarges when the working plunger is being moved forward by the compressed gas out of its starting position in the direction away from the rear side. What this achieves is that an impact or a major force for acceleration of the control plunger can be applied via the catch element such that the compressed gas motor cannot come to a standstill due to a resting or blocked control plunger. For the same purpose, the invention can provide the mass of the working plunger, and in particular of the working plunger with the catch element firmly connected to it, to be larger than the mass of the control plunger.

Moreover, the invention proposes the restoring element to be an elastic spring that is arranged in the internal space between the working plunger and a front end of the internal space, whereby the front end of the internal space is arranged opposite from the rear side of the internal space.

This provides a compressed gas motor that is particularly simple and inexpensive to manufacture and works well regardless of its orientation.

The invention can just as well provide the control plunger, in the first position, to be pushed or pulled over the working plunger against the rear side by the restoring element, whereby the gas inlet opening exits into an opening or into the intervening space between the working plunger and the control plunger.

This structure allows the compressed gas motor to be brought into a starting position (the starting position of the working plunger and control plunger) in a situation, in which no compressed gas is supplied through the gas inlet opening into the internal space, from which it starts when a compressed gas is supplied through the gas inlet opening.

According to a preferred refinement, the invention can just as well provide at least one gas-permeable passage to be arranged in the control plunger and to connect the front side of the control plunger facing the working plunger to the rear side of the control plunger facing the rear side of the internal space. The connection is a gas-permeable connection.

The control plunger can then touch against the internal walls of the internal space by its entire circumference. This ensures a stable linear guidance of the control plunger in the internal space.

Preferred embodiments of the invention can be characterised in that the working plunger touches against the internal wall of the internal space by its entire circumference, preferably touches by its entire circumference in gas-tight and pressure-tight manner against the internal space by means of a sealing element.

This ensures that the compressed gas does not flow past the working plunger to the effect that the performance or power of the compressed gas motor is reduced.

For compressed gas motors according to the invention for producing a mechanical motion, the invention can preferably provide a rod, in particular a plunger rod, to be attached to the side of the working plunger that faces away from the control plunger and projects from the internal space, preferably projects through the front side of the internal space.

In this variant, the rod produces a periodical push. Through the use of a plunger rod that is connected to the working plunger by means of an axis, the periodical motion of the working plunger can be converted into a rotary motion. For this purpose, the plunger rod preferably is or can be connected to a crankshaft.

In this variant, the invention can just as well provide the rod to be guided through a gas-tight feed-through through the front side of the internal space out of the internal space and can provide the internal space to be closed in gas-tight and pressure-tight manner between the front side and the working plunger and to form a gas-operated spring as restoring element.

This dispenses with the need to have a separate restoring element or the gas-operated spring can be used as additional restoring element. In the former case, the compressed gas motor can be made very inexpensively, whereas a stronger restoring force can be attained in the latter case and the motor can withstand failure or deterioration of one of the restoring elements.

In variants, which are intended instead for direct ejection of a liquid, the invention can provide an ejection opening in the front side of the internal space opposite from the rear side, and can provide a liquid supply opening to be arranged in the front side and/or in the lateral wall of the internal space and to not be covered by the working plunger at least for part of the time and, in the non-covered state, to be arranged between the working plunger and the front side of the internal space.

Due to this structure, the swept volume of the working plunger is used, at least in part, to take up a liquid and/or to take up a mixture of gas and liquid. When the working plunger is being moved in the direction of the front side of the internal space, the liquid, and/or the liquid-gas mixture, is ejected through the ejection opening. The compressed gas motor can then be used directly for lavage systems.

In this embodiment, the invention can provide the ejection opening to be connected to the surroundings by a valve element, in particular a lip valve, whereby the valve element is open in the presence of sufficient over-pressure as compared to the ambient pressure and is closed otherwise, and can provide a tube or a hose with a non-return valve to be connected at the liquid supply opening and to open in the presence of an under-pressure in the internal space between the working plunger and the front side of the internal space and to thus enable liquid to be supplied into the internal space.

This allows the internal space to be filled automatically with a liquid. As a result, a particularly simple and inexpensive structure of a compressed gas motor for a lavage system is attained. With regard to the definition of a sufficient pressure, reference shall be made to known lip valves for the same or similar purposes.

Compressed gas motors according to the invention can also be characterised in that the invention provides the catch element to be a string, a cable, a thread, a chain or an elastic spring that is attached to the working plunger and to the control plunger or the catch element to comprise a rod, a string, a cable, a thread, a chain or an elastic spring that is attached to the working plunger or to the control plunger and has a catch attached to it that engages a projection in the working plunger or in the control plunger, whereby the catch element preferably is provided by a rod that is attached to the working plunger and extends through a feed-through in the control plunger and has a catch attached to it that does not fit through the feed-through in the control plunger and engages the control plunger on the rear side thereof in order to pull the control plunger along, when the working plunger is sufficiently far away from the control plunger for this purpose and moves in the direction away from the control plunger.

Said catch elements can be used to attain an increase in the distance between the working plunger and the control plunger in structurally simple manner before the control plunger is being pulled along by the working plunger. By this means, the efficiency of the motor can be improved and the risk of a standstill of the motor can be decreased.

Moreover, the invention can preferably provide the working plunger to comprise two differently-sized cross-sectional surfaces perpendicular to the motion direction of the working plunger, whereby the internal space comprises matching internal walls with different cross-sectional surfaces and the cross-sectional surface on the side of the working plunger facing the rear side is larger than the cross-sectional surface of the opposite front side of the working plunger. In this context, the invention can preferably provide the cross-sectional surface on the side of the working plunger facing the rear side to be no more than half the size of the cross-sectional surface of the opposite front side of the working plunger.

This is advantageous in that the consumed gas volume is made smaller through the decrease in working space, i.e. space, in which the work is performed on the working plunger by the compressed gas, than if both parts of the working plunger were of equal size. This reduces the gas consumption of the compressed gas motor. Accordingly, a smaller amount of compressed gas has to be fed through the compressed gas motor per working cycle. It is particularly preferable in this variant to provide the control plunger to have the same small cross-sectional surface as the side of the working plunger facing the rear side. Moreover, it is preferred in this embodiment that the side of the working plunger facing the rear side having the smaller diameter is longer, in terms of the length in the motion direction of the working plunger, than the side of the working plunger facing the front side, particularly preferably is at least twice as long as the side of the working plunger facing the front side. Moreover, in this embodiment, the invention can provide a full-circumference seal, which seals the working plunger with respect to the internal walls of the internal space, to be arranged about the working plunger, about both sides of the working plunger for each of the two cross-sectional surfaces.

Referring to oval or circular cross-sectional surfaces of the working plunger, it is always possible to refer to a radius, diameter or cross-section rather than a cross-sectional surface. Accordingly, referring to oval or circular cross-sectional surfaces of the working plunger or other cross-sectional surfaces of the working plunger to which a cross-section, radius or diameter is applicable, the invention can provide the working plunger to comprise two differently-sized diameters or cross-sections perpendicular to the motion direction of the working plunger, whereby the internal space comprises matching internal walls having different diameters or cross-sections and the diameter or cross-section on the side of the working plunger facing the rear side is smaller than the diameter or cross-section of the opposite front side of the working plunger. In this context, the invention can preferably provide the diameter or cross-section on the side of the working plunger facing the rear side to be no more than half the size of the diameter or cross-section of the opposite front side of the working plunger.

The objects of the invention with regard to a lavage system are met by a lavage system comprising at least one compressed gas motors of this type, in which the compressed gas motor or compressed gas motors allow(s) can be used to generate a periodical spray puff of a liquid.

The lavage system can be provided such that it comprises a connector for a compressed gas cartridge, a pressure reducer, an evaporation space for evaporation of liquid residual liquid gas, and a manually actuated control valve, whereby the evaporation space is connected to the connector by means of a compressed gas line, the pressure reducer is connected to the evaporation space by means of a compressed gas line, the control valve is connected to the pressure reducer by means of a compressed gas line, and the gas inlet opening of the compressed gas motor is connected to the control valve by means of a compressed gas line, and whereby, preferably, a liquid supply opening of the compressed gas motor is connected to a liquid line.

The objects of the invention are also met by the use of a compressed gas motor of this type as motor for a lavage system, a rapping motor, as motor for a toy, a vibration motor, as drive for a dosing facility, as shaker motor or as pump, in particular as lubricant pump.

The underlying objects of the invention are also met by a method for generating a periodical motion using a compressed gas, in particular through the use of a compressed gas motor of this type, in which

A) the working plunger and the control plunger, in a starting state, are situated in the internal space such as to be at a first distance from each other, whereby the control plunger closes the gas outlet opening and the gas inlet opening between the working plunger and the control plunger is open;
B) the compressed gas is supplied into the internal space between the working plunger and the control plunger;
C) the pressure of the compressed gas accelerates and moves the working plunger in the direction of a front side of the internal space away from the control plunger, whereby the distance between working plunger and control plunger increases;
D) the control plunger is pulled along by the working plunger by means of the catch element, preferably as soon as the second distance is reached;
E) the control plunger closes the gas inlet opening due to the motion of the control plunger;
F) the control plunger opens the gas outlet opening due to the motion of the control plunger;
G) the restoring element accelerates the working plunger in the direction of the rear side of the internal space;
H) the compressed gas flows between the working plunger and the control plunger through at least one gas-permeable opening through the control plunger into the space between the rear side of the control plunger and the rear wall of the internal space;
I) the compressed gas in the space between the rear side of the control plunger and the rear wall of the internal space is released to the surroundings through the open gas outlet opening;
J) the working plunger hits against the control plunger and moves it in the direction of the rear side of the internal space;
K) the gas outlet opening is closed again by the reverse motion of the control plunger; and
L) the reverse motion of the control plunger opens the gas inlet opening again such that compressed gas is again supplied into the internal space between working plunger and control plunger.

The working plunger moves in step J) because it is being accelerated by the restoring element. The compressed gas, in contrast, no longer exerts a force on the working plunger when the working plunger hits against the control plunger, since the pressure and/or the compressed gas has escaped already through the open gas outlet opening.

The steps proceed in logical, to some extent chronological, order, whereby the periods of time, for which the steps according to the invention proceed, can overlap in part and in time. Preferably, the motor is restored to its starting state by the restoring element. However, the compressed gas motor also works if the starting state is reached again only once no compressed gas is available any longer at the gas inlet opening.

The compressed gas is supplied at a pressure of at least 150 kPa, preferably at a pressure of at least 300 kPa. According to the invention, a CO₂ cartridge containing liquid CO₂ is used as the source of compressed gas. It is particularly preferred according to the invention to reduce the pressure from the CO₂ cartridge by means of a pressure reducer (pressure-reducing valve) to a level between 150 kPa and 500 kPa before feeding it through the gas inlet opening into the compressed gas motor.

In methods according to the invention, the invention can provide the cycle to repeat when the feeding of the compressed gas is repeated.

Moreover, the invention can provide the first distance between working plunger and control plunger to be adjusted by means of at least one spacer and the second distance between working plunger and control plunger to be adjusted by a catch element, preferably by means of the length of the catch element.

Preferably, the second distance is selected to be at least twice the first distance.

The underlying objects of the invention are also met by a method for producing a spray puff comprising the afore-mentioned procedural steps, whereby, upon a motion of the working plunger away from the rear side of the internal space, a liquid or a liquid-gas mixture is ejected from the space between the working plunger and the front side of the internal space through an ejection opening on the front side of the internal space, and upon a motion of the working plunger towards the rear side of the internal space, a liquid or a liquid-gas mixture is pushed or sucked through a liquid supply opening into the space between the working plunger and the front side of the internal space.

In this method, the invention can provide, upon the motion of the working plunger towards the front side of the internal space, the pressure in the space between the working plunger and the front side of the internal space to open and/or keep open a valve at the ejection opening and to close and/or keep closed a non-return valve connected to the liquid supply opening, and, upon the motion of the working plunger towards the rear side of the internal space, the lesser pressure in the space between the working plunger and the front side of the internal space to close and/or keep closed the valve on the ejection opening and to open and/or keep open the non-return valve connected to the liquid supply opening.

The invention is based on the surprising finding that having a working plunger and a control plunger with a variable distance from each other allows a simple and reliably running compressed gas motor to be provided that has no dead centre at which the compressed gas motor comes to a standstill without being able to re-start again. Said “dead centre” is prevented according to the invention in that a defined state of the valves is feasible in the compressed gas motor at all times. Defined state shall be understood to mean that a valve is either open or closed. It can be ensured through appropriate adjustment of the two distances of the two plungers with respect to each other during operation of the compressed gas motor that the compressed gas motor can be utilised as appropriate for the respective purpose. In particular, using the compressed gas motor as a pump for generating spray puffs of a liquid, the compressed gas motor can be designed in simplest manner in that the swept volume of the working plunger is used directly as the pump.

The working plunger and the control plunger do not move concurrently while keeping a constant distance in this respect. The working plunger moves first. Once the working plunger reaches a certain distance from the control plunger and thus reaches a certain velocity, it pulls the control plunger along all of a sudden in that the cables, strings or chains of the catch element are tensioned suddenly or in that the catch hits against the projection of the control plunger or of the working plunger and thus causes the control plunger to be carried along. Accordingly, the rationale underlying the present invention, in particular, is to decouple, in time, the valve control by means of the control plunger from the motion of the working plunger. This surprisingly allows a forceful motion of the working plunger to be attained. A forceful motion of the working plunger results also by comparison to the compressed gas motor according to WO 2012/038003 A1 having the two-part, but firmly connected working plunger, such that more force can be transmitted upon each motion of the working plunger.

This allows the front part of the hollow space to be used as pump space directly and without a gear or a transmission for reinforcement. The spray puff of a lavage system can thus be generated directly by means of the working plunger. This allows energy losses during the pumping process to be prevented. It is therefore excluded that the gear or part of the transmission might become defective. Moreover, the structure becomes more compact and less expensive as compared to when these parts have to be attached or installed.

In this context, the invention is based on the following rationale. A working plunger, which can be shifted in axial direction, is pushed against a control plunger in a hollow cylinder by a restoring element. A gas inlet opening and a gas outlet opening are arranged separate from each other in the jacket surface of the hollow cylinder and are connected to the internal space of the hollow cylinder in gas-permeable manner. The gas inlet opening is situated in the direction of the working plunger and restoring element and the gas outlet opening is situated opposite with respect to these in the direction of the gas-impermeable closure of the hollow cylinder. The control plunger can be shifted in axial direction in the hollow cylinder and, depending on its position, can close either just the gas inlet opening, while the gas outlet opening is open at this time, or close just the gas inlet opening, while the gas outlet opening is open at this time. The control plunger comprises a passage that extends axially through the control plunger.

The front side of the working plunger opposite from the restoring element has a catch pin situated on it that has a catch arranged on its end. The catch pin is mobile in axial direction in the hollow space of the control plunger. A limit stop is situated on the control plunger in the direction of the front side of the working plunger, on which the catch pin is arranged. The catch can engage said limit stop upon axial motion of the catch pin due to the motion of the working plunger in the direction of the restoring element, and move the control plunger in the direction of the restoring element upon the axial motion. A hollow space is situated between working plunger and control plunger and is maintained by at least one spacer, when the working plunger and the control plunger touch against each other via the spacer, and keeps the working plunger at a distance from the control plunger. In the absence of applied compressed gas, said hollow space is situated right above the gas inlet opening.

A compressed gas motor according to the invention for this purpose can, for example, be composed of:

a) a hollow cylinder that possesses at least one gas inlet opening and one gas outlet opening that are arranged separately from each other on the jacket surface of the hollow cylinder;
b) a working plunger that is arranged in the hollow cylinder such as to be mobile in axial direction;
c) an elastic restoring element that is arranged in the hollow cylinder and engages a front face side of the working plunger;
d) a catch pin that is connected to the working plunger on the front side of the working plunger opposite from the restoring element;
e) a catch that is connected to the catch pin;
f) a control plunger that can be shifted in axial direction in the hollow cylinder and has a length in axial direction that is at least equal to the sum of the axial distance between the gas inlet opening and the gas outlet opening and the axial length of the gas inlet opening and/or of the gas outlet opening, whereby the control plunger, shifted axially over the gas inlet opening, closes the gas inlet opening in gas-tight manner and the gas outlet opening is open at the same time, the control plunger, shifted over the gas outlet opening, closes the gas outlet opening in gas-tight manner and the gas inlet opening is open at the same time, the control plunger possesses at least one hollow space, in which the catch pin can be shifted in axial direction;
g) a limit stop on the control plunger that is arranged appropriately on the side of the control plunger facing the working plunger, such that a motion of the working plunger in the direction of the restoring element moves the catch pin through the hollow space of the control plunger and then the catch arranged on the catch pin hits against the limit stop and moves the control plunger in the direction of the restoring element if the working plunger moves further;
h) a limit stop that limits the axial motion of the control plunger opposite to the restoring element; and
i) a gas-impermeable closure on the end of the hollow cylinder opposite from the restoring element such that a hollow space is formed by the hollow cylinder that is bounded by the gas-impermeable working plunger and the gas-permeable control plunger.

The working principle of the compressed gas motor is that compressed gas is first made to enter through the gas inlet opening, which is open in the standby state, into the hollow space (internal space). The compressed gas propels the working plunger in the direction of the restoring element. In the process, the catch pin and catch move along on the inside of the hollow space of the control plunger without the position of the control plunger being changed. Following further axial motion of the working plunger in the direction of the restoring element, the catch hits against the limit stop of the control plunger. Then the catch takes the control plunger along in the direction of the restoring element, whereby the gas inlet opening is being closed by the shifted control plunger and right afterwards the gas outlet opening is being opened. It is essential in this context that the control plunger has an appropriate axial extension such that it is impossible for geometrical reasons that the gas inlet opening and the gas outlet opening are both open at the same time. Due to the inertia of the moving working plunger, the so-called “dead centre” is passed without needing flywheels or similar devices or inertial masses for this purpose. Once the gas outlet opening is open, the at least partially relaxed gas escapes towards the surroundings. As a result, the pressure behind the working plunger decreases. As soon as the restoring force is larger than the pressure force of the residual at least partially relaxed gas that is still present and acts on the working plunger surface, the working plunger is moved back into the starting position by the restoring element, whereby the working plunger also pushes the control plunger back into the starting position. The catch pin also moves along axially inside the hollow space of the control plunger, whereby the catch is being detached from the limit stop and also returns to its starting position. The hollow space between working plunger and control plunger is situated above the gas inlet opening again. Then, the process just described commences again and repeats for as long as compressed gas is applied to the gas inlet opening.

The working plunger can be shifted axially in the hollow cylinder in gas-tight manner. Sealing can be achieved by O-rings or by a fit that is not too tight.

The control plunger can be shifted axially in the hollow cylinder, whereby, depending on the position of the control plunger in the hollow cylinder, either the gas inlet opening or the gas outlet opening is being closed in gas-tight manner, whereby either just the gas inlet opening or just the gas outlet opening is open at any given time. Preferably, only the gas inlet opening or the gas outlet opening can be open at any given time in order to prevent “short-circuiting” in terms of the flow of compressed gas.

At least one spacer is arranged on the working plunger and/or on the control plunger and keeps the control plunger at a distance from the working plunger, whereby it is preferred according to the invention to have the minimal distance between the control plunger and the working plunger be at least 0.1 mm. Said minimal distance is required to allow the compressed gas to enter into said hollow space (and/or intervening space or gap) and to move the working plunger while the volume of the hollow space is being increased.

It is important to note that, without the effect of the compressed gas, but due to the pressure of the restoring element acting on the working plunger, the working plunger is positioned appropriately such that the hollow space between the control plunger and the working plunger that is formed in the hollow cylinder by the at least one spacer is connected in gas-permeable manner just to the gas inlet opening, whereby the gas outlet opening is closed by the control plunger at this time. This means that the hollow space, in the starting position of the compressed gas motor without compressed gas being applied, is always situated right above the gas inlet opening such that the hollow space can be enlarged by moving the working plunger when compressed gas is being applied. This ensures automatic start-up of the compressed gas motor from a defined starting position (the original position) of the working plunger and control plunger.

For the compressed gas motor to work properly, it is necessary that the control plunger possesses at least one axial gas feed-through (and/or a passage) that connects the hollow space, which is formed by the hollow cylinder, the front side of the working plunger, and the control plunger, in gas-permeable manner to the hollow space formed by the opposite front side of the control plunger, the hollow cylinder, and the gas-impermeable closure. After closure of the gas inlet opening, this allows the compressed gas to get to the open gas outlet opening and to escape into the surroundings. A pressure release is feasible by this means such that the thus decreasing gas pressure allows the restoring element to push the working plunger and the control plunger back into their starting position, whereby the gas inlet opening is being opened and the gas outlet opening is being closed. The drive process then starts up again. Repeating this process periodically generates a periodical linear motion of the working plunger. The process proceeds automatically without any influence of external valves as long as compressed gas is being applied.

According to the invention, it is particularly preferred to use coil springs and leaf springs as restoring element. In the scope of the invention, these can be made of steel, stainless steel or plastic materials. It is essential that the restoring element can drive the working plunger and the control plunger back into the starting position after the gas has been released.

Accordingly, the invention can provide the compressed gas motor to be manufactured from thermoplastic materials with polypropylene, polyethylene, polyamide-6, and polyamide-12 being particularly preferred. In addition, all common plastic materials in this technology are well-suited.

A method according to the invention for generating a periodical linear motion by means of a compressed gas motor according to the invention can be implemented, for example, in that

a) the working plunger is first pushed appropriately in the direction of the limit stop (the rear side and/or rear panel) by the restoring element such that the control plunger touches against the limit stop, whereby the gas inlet opening is open and the gas outlet opening is closed in gas-tight manner by being overlapped by the control plunger;
b) compressed gas flows in through the open gas inlet opening and moves the working plunger in the direction of the restoring element, whereby the catch pin arranged on the rear side of the working plunger is also moved along through the axial hollow space of the control plunger in the direction of the restoring element until the catch reaches the limit stop on the control plunger;
c) then the catch engages the limit stop of the control plunger and, upon further motion of the working plunger in the direction of the restoring element, shifts the control plunger axially in the direction of the restoring element by means of which the gas inlet opening is being closed by the control plunger and the gas outlet opening is being opened;
d) the compressed gas then flows through the open gas outlet opening from the hollow cylinder into the surroundings until the gas pressure is so low that the restoring force of the restoring element is larger than the force of the residual compressed gas remaining in the hollow cylinder, by means of which the restoring element shifts the working plunger in the direction of the control plunger, by means of which the working plunger also shifts the control plunger axially opposite to the restoring element by means of the at least one spacer, by means of which the gas inlet opening is being opened and the gas outlet opening is being closed by the control plunger, and the process is repeated afterwards.

The process is repeated for as long as compressed gas is fed inside through the gas inlet opening.

It is essential to the invention that the periodical linear motion of the working plunger through the compressed gas motor is triggered independently by the action of compressed gas without any influence of external valves.

The generation of periodical linear motions by means of the compressed gas motor necessitates a compressed gas that has a pressure of more than or equal to 0.5 bar (50 kPa) with respect to the surrounding atmosphere.

Moreover, the invention can preferably provide carbon dioxide, air, nitrogen, dinitrogen monoxide or mixtures thereof to be used as compressed gas. It is particularly advantageous in this context to use liquid carbon dioxide which is commercially available in metal cartridges at low cost. The use of liquid carbon dioxide allows large gas volumes to be accommodated in space-saving manner in relatively small metal cartridges. As a result, these cartridges can be accommodated in hand-pieces of lavage systems (and/or hand-held lavage systems). In addition, other compressed gases can be used just as well, including water vapour and mixtures of water vapour, carbon dioxide, carbon monoxide.

Another exemplary embodiment of the invention is a compressed gas-driven liquid pump having a compressed gas motor according to the invention, composed of

a) a hollow cylinder that possesses at least one gas inlet opening and one gas outlet opening that are arranged separately from each other on the jacket surface of the hollow cylinder;
b) a working plunger that is arranged in the hollow cylinder such as to be mobile in axial direction;
c) a hollow space formed by the jacket surface of the hollow cylinder, the front side of the working plunger, and the liquid-tight front surface of the hollow cylinder, whereby said hollow space is connected to a non-return valve in liquid-permeable manner, whereby the hollow space is connected to an outlet valve in liquid-permeable manner;
d) an elastic restoring element that is arranged in the hollow cylinder and engages a front side of the working plunger, and rests against the liquid-tight front surface of the hollow cylinder;
e) a catch pin that is connected to the working plunger on the front side of the working plunger opposite from the restoring element;
f) a catch that is connected to the catch pin;
g) a control plunger that can be shifted in axial direction in the hollow cylinder and has a length in axial direction that is at least equal to the sum of the axial distance between the gas inlet opening and the gas outlet opening and the axial diameter of the gas inlet opening and of the gas outlet opening, whereby the control plunger, shifted axially over the gas inlet opening, closes the gas inlet opening in gas-tight manner and opens the gas outlet opening at the same time, the control plunger, shifted over the gas outlet opening, closes the gas outlet opening in gas-tight manner and opens the gas inlet opening at the same time, the control plunger possesses at least one hollow space, in which the catch pin can be shifted in axial direction;
h) a limit stop on the control plunger that is arranged on the side of the control plunger facing the working plunger, such that a motion of the working plunger in the direction of the restoring element moves the catch pin through the hollow space of the control plunger, and then such that the catch arranged on the catch pin hits against the limit stop and moves the control plunger in the direction of the restoring element upon the working plunger moving further;
h) a limit stop that limits the axial motion of the control plunger opposite to the restoring element; and
i) a gas-impermeable closure on the end of the hollow cylinder opposite from the restoring element such that a hollow space is formed by the hollow cylinder that is bounded by the working plunger and the gas-impermeable closure.

The compressed gas motor can also be used as a drive of pumps, preferably as a drive of medical lavage systems, as a drive of drug dosing pumps, as a drive of lubricant pumps, as a drive of dosing pumps for pesticides, as a drive of dosing pumps for laundry detergents, and particularly preferably to drive medical lavage systems intended for single use only. In addition, the compressed gas motor can also be used to generate rotary motions after connecting the working plunger to suitable mechanical coupling elements, such as plunger rods. Moreover, the compressed gas motor can be used to drive moved machine elements. It is also feasible to use the compressed gas motor to drive toy vehicles and toys.

A medical lavage system for single use, in which the compressed gas motor according to the invention is used, may be structured, for example, as follows: A gas cartridge for liquefied compressed gas, an opening facility for the compressed gas cartridge, a gas-tight connection of the opening facility to an evaporation container for the liquefied compressed gas, a gas discharge line from the evaporation container connected to a pressure-reducing valve that is connected, by means of another gas line, to a control plunger that can regulate the gas flow and in turn is connected to the pump driven by the compressed gas motor to which, in turn, a liquid dispensing tube is connected. With the exception of the liquid dispensing tube, all elements of the lavage system can be arranged in the hand-piece of the system and are easy to hold by hand.

Exemplary embodiments of the invention shall be illustrated in the following on the basis of thirteen schematic figures, though without limiting the scope of the invention. In the figures:

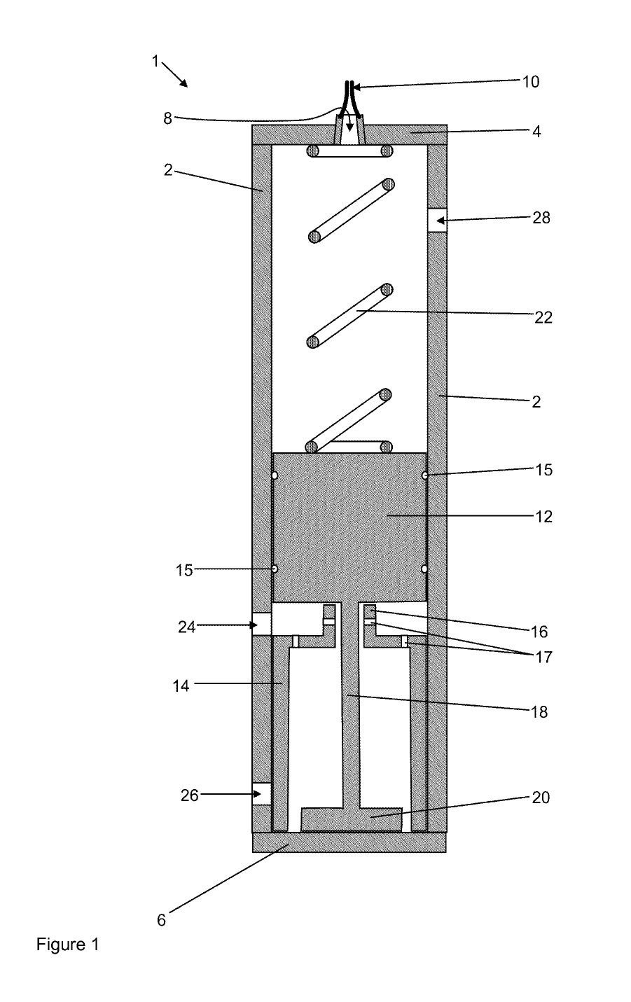

FIG. 1: shows a schematic cross-sectional view of a compressed gas motor according to the invention in the starting state and/or the starting position;

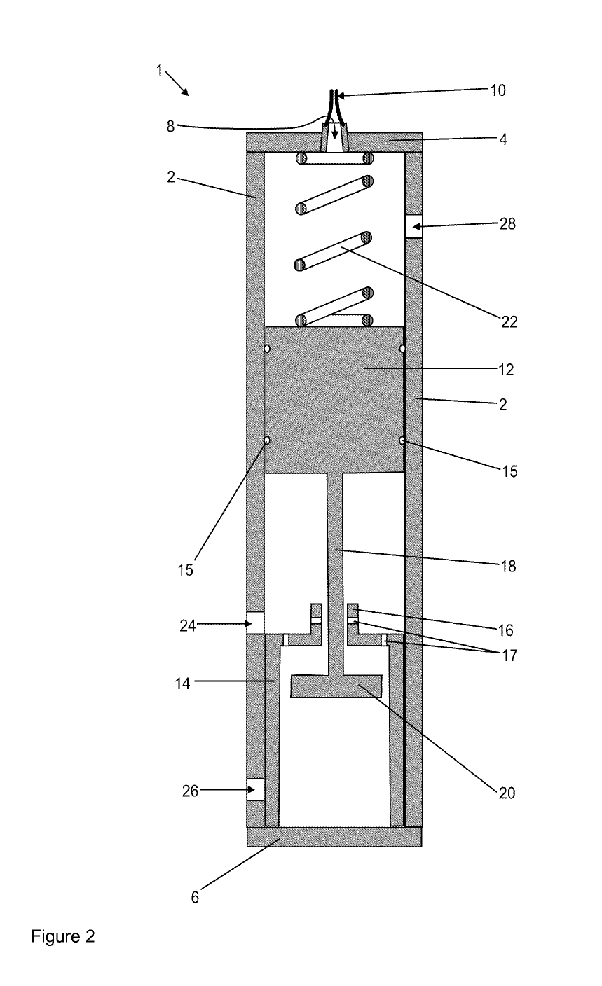

FIG. 2: shows a schematic cross-sectional view of the inventive compressed gas motor according to FIG. 1, in which the working plunger is moved by the compressed gas;

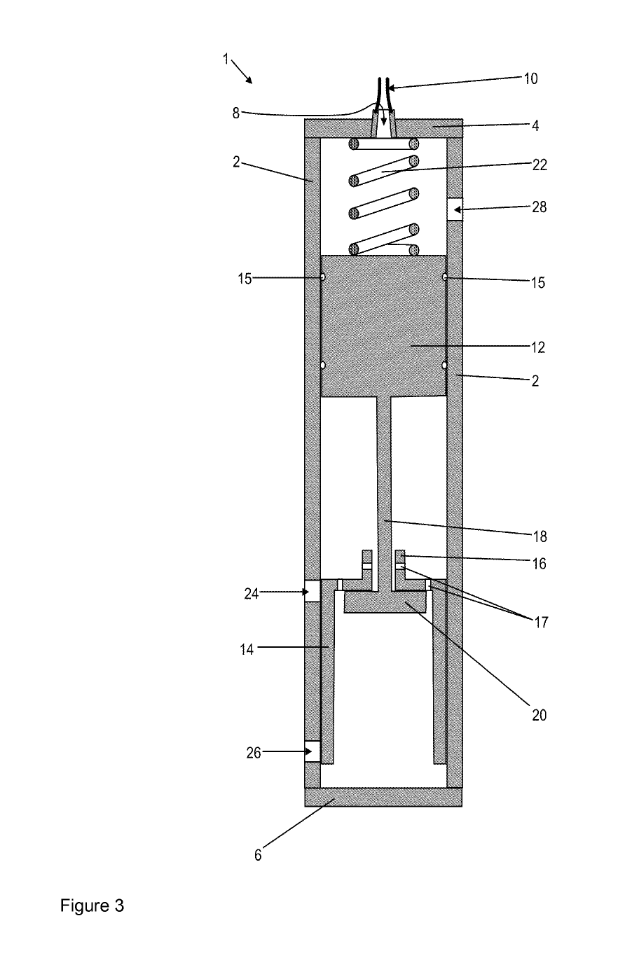

FIG. 3: shows a schematic cross-sectional view of the inventive compressed gas motor according to FIG. 1, in which the catch of the working plunger engages the control plunger and pulls it along;

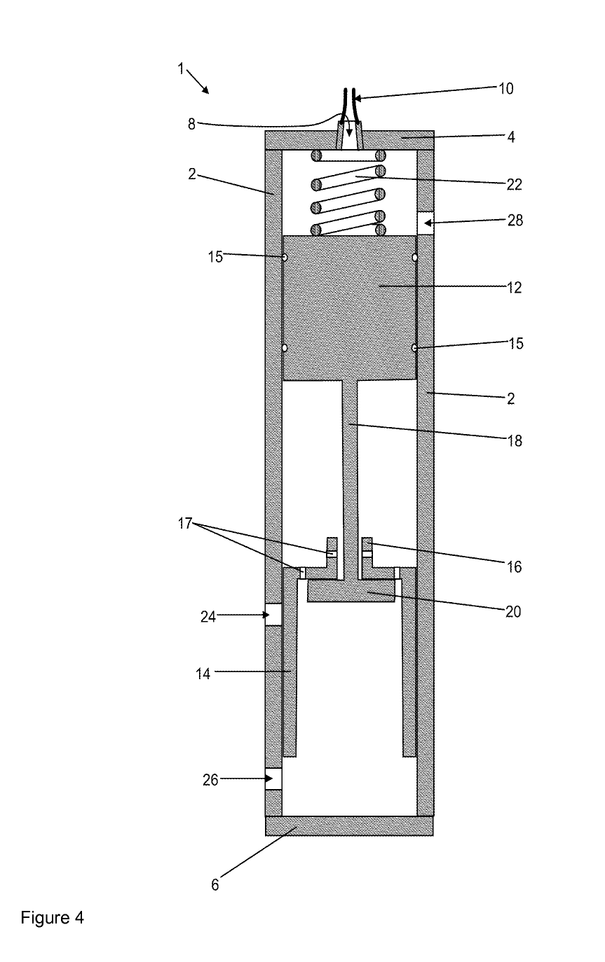

FIG. 4: shows a schematic cross-sectional view of the inventive compressed gas motor according to FIG. 1, in which the working plunger and the control plunger are deflected maximally in the direction of the front side;

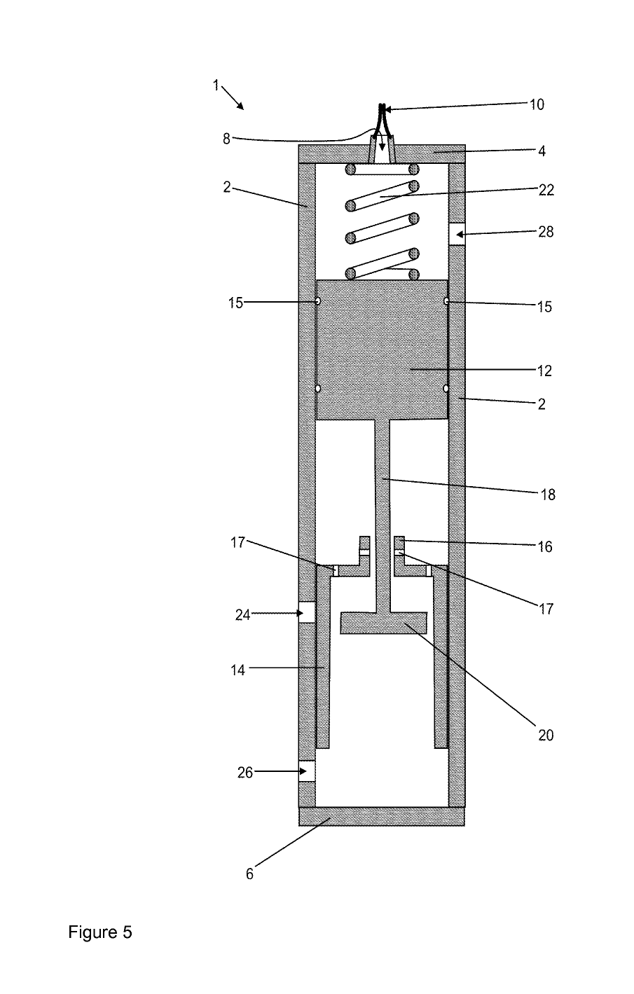

FIG. 5: shows a schematic cross-sectional view of the inventive compressed gas motor according to FIG. 1, in which the working plunger is again moved in opposite direction by the spring;

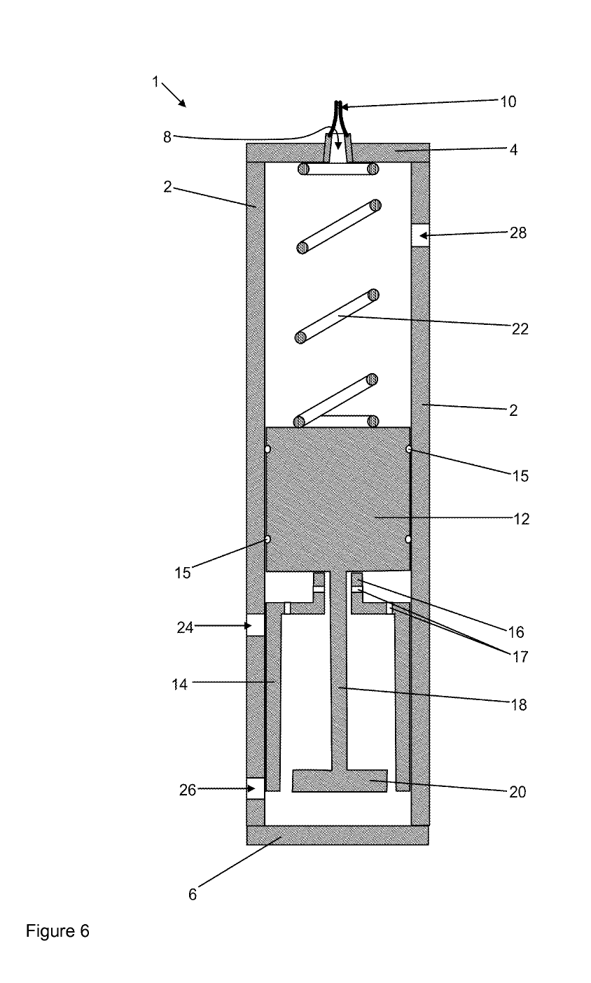

FIG. 6: shows a schematic cross-sectional view of the inventive compressed gas motor according to FIG. 1, in which the working plunger hits against the control plunger again and moves it in the opposite direction;

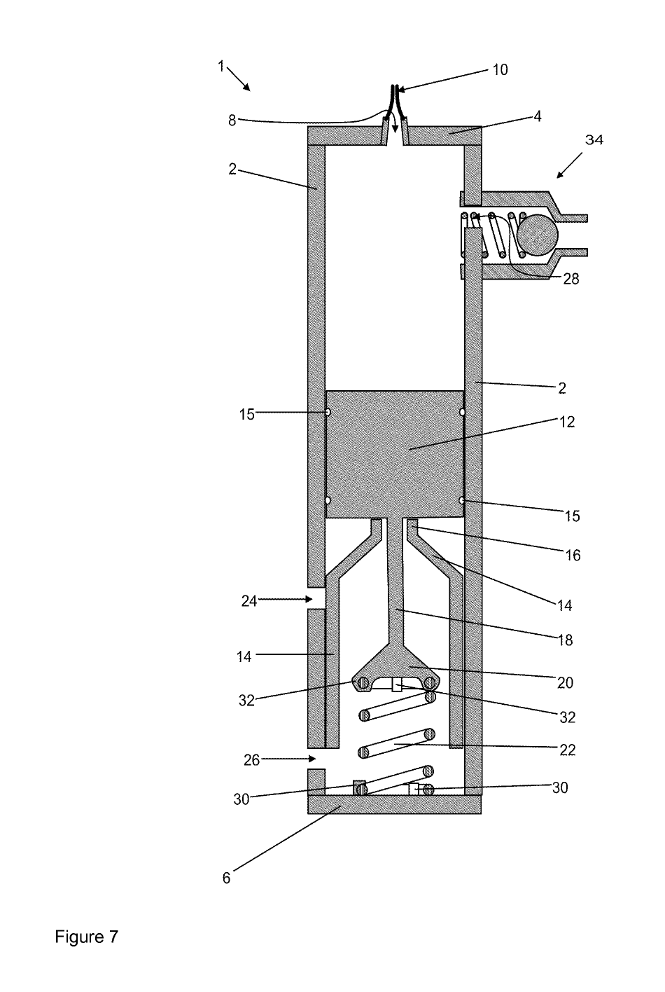

FIG. 7: shows a schematic cross-sectional view of an alternative inventive compressed gas motor having a tension spring as restoring element and a non-return valve on the liquid supply opening;

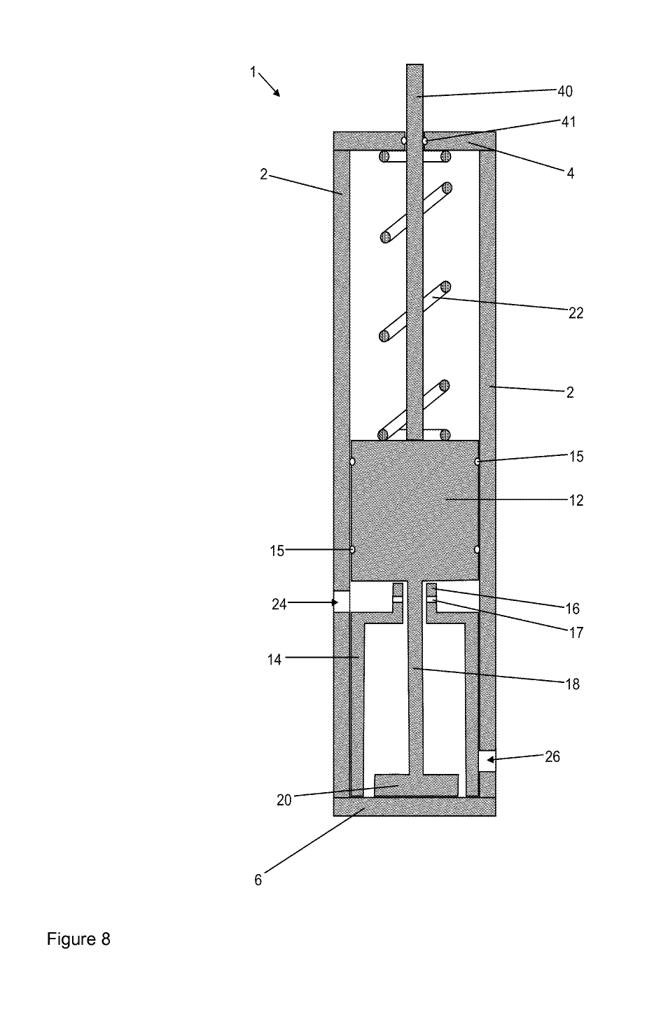

FIG. 8: shows a schematic cross-sectional view of another alternative inventive compressed gas motor having a rod for forming a push-type motor;

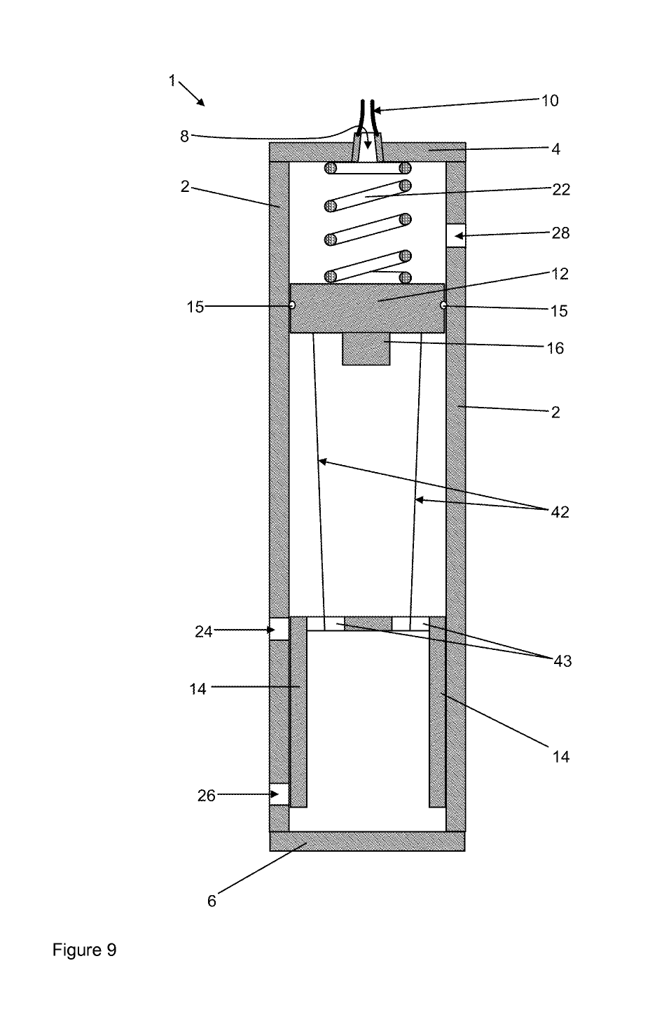

FIG. 9: shows a schematic cross-sectional view of a fourth alternative inventive compressed gas motor having a string or a cable as catch element;

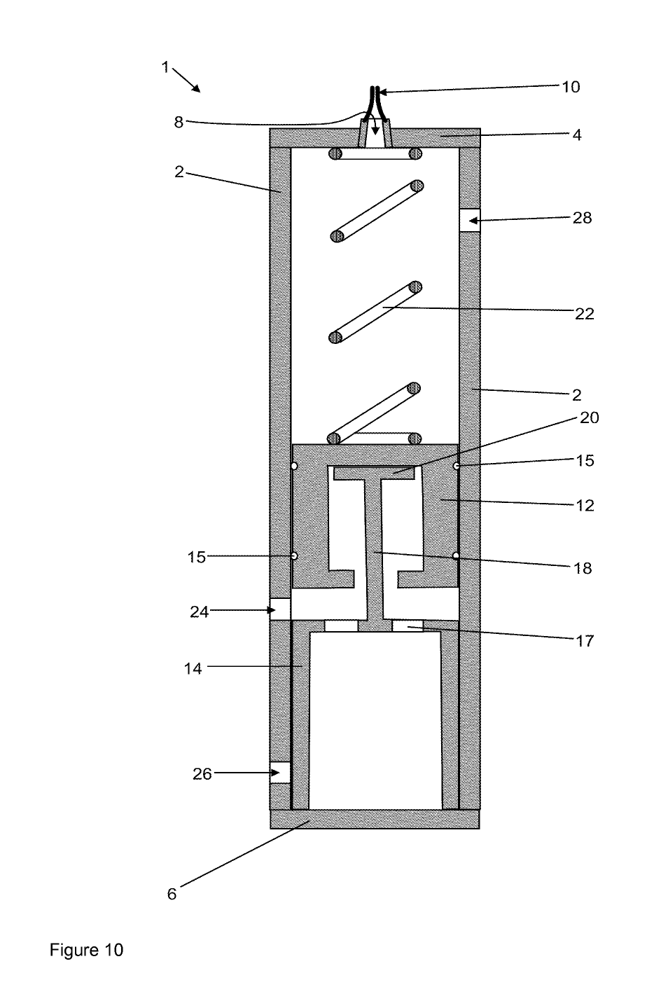

FIG. 10: shows a schematic cross-sectional view of a fifth alternative inventive compressed gas motor, in which the catch element is fastened to the control plunger;

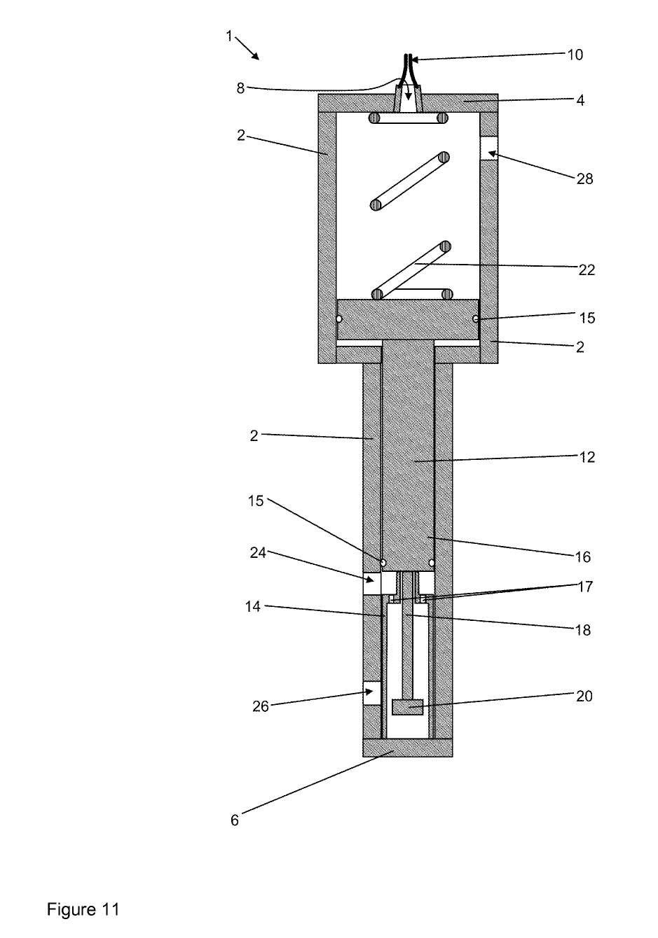

FIG. 11: shows a schematic cross-sectional view of a sixth alternative inventive compressed gas motor having a smaller working space;

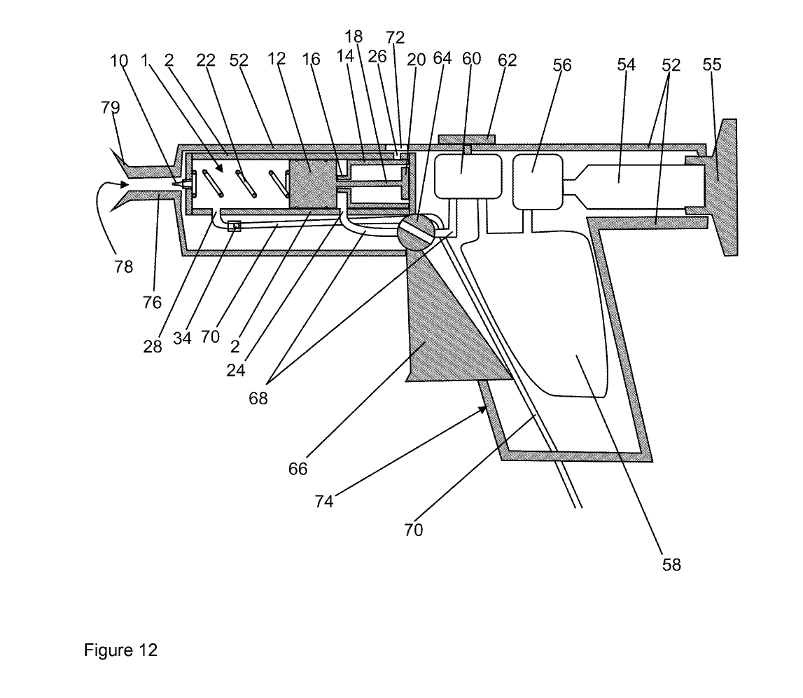

FIG. 12: shows a schematic cross-sectional view of an inventive lavage system having a compressed gas motor according to the invention; and

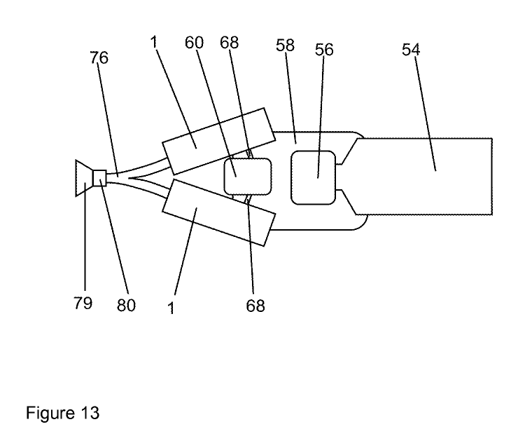

FIG. 13: shows a schematic top view onto an inventive lavage system having two inventive compressed gas motors.

To some extent, identical or similar components are identified in the figures through the same reference numbers.

FIGS. 1 to 6 show schematic cross-sectional views of an inventive compressed gas motor 1 in chronological order during a working cycle. The cross-sections contain the symmetry axis of the components of the compressed gas motor 1, i.e. the cross-section cuts through the middle. The compressed gas motor 1 comprises a hollow body 2 that is made of plastic material and has a cylindrical internal space. The internal space is closed through a cover plate 4 on the front side and through a rear plate 6 on the rear side. An ejection opening 8 for dispensing a jet of liquid is provided in the cover plate 4.

The ejection opening 8 is closed by means of a lip valve 10, which opens when a liquid is ejected from the internal space, i.e. when the pressure in the internal space is sufficiently high. This means that the pressure in the internal space is higher than the ambient pressure plus the elastic force of the lip valve 10. The rear plate 6 closes the internal space of the hollow body 2 in gas-tight and pressure-tight manner. The internal space contains a working plunger 12 that is arranged such as to be mobile along the cylinder axis of the cylindrical internal space, and a control plunger 14 that is mobile in the same manner. The control plunger 14 touches tightly against the cylinder walls of the internal space by its entire circumference. The working plunger 12 is sealed with respect to the internal walls of the internal space by means of two sealing rings 15 made of rubber or another elastic material. For this purpose, the sealing rings 15 touch by their entire circumference against the working plunger 12 and the internal walls of the hollow body 2.

The control plunger 14 is also made of plastic material and has the basic shape of a tube with a cover. A tube-shaped spacer 16 is arranged on the cover and points in the direction of the working plunger 12 and projects beyond the walls of the tube of the control plunger 14 that touches against the internal walls of the internal space. Passages 17 are arranged in the cover and spacer 16 and are to enable gas exchange between the two sides of the control plunger 14. Another central passage on the symmetry axis is provided within the spacer 16.

The control plunger 14 is arranged between the working plunger 12 and the rear plate 6 such as to be mobile in the internal space. A catch unit consisting of a catch pin 18 and a catch 20 is fastened to the working plunger 12. The catch pin 18 and the working plunger 12 are preferably provided as a single part and the catch pin 18 is also made of plastic material. The catch pin 18 is a cylindrical rod that extends through the axial passage in the control plunger 14. The catch 20 is a flat disc that does not fit through the central passage in the spacer 16 of the control plunger 14.

A steel spring 22 is arranged in the internal space between the cover plate 4 and the working plunger 12. The steel spring 22 pushes the working plunger 12 onto the spacer 16 on the control plunger 14 such that the control plunger 14 is held in the position shown when no compressed gas (a gas at a pressure above ambient pressure) is fed through a gas inlet opening 24 into the internal space. The steel spring 22 does not have to touch against the working plunger 12 and/or the cover plate 4. It is also sufficient if the steel spring 22 is sufficiently long such that it ensures a sufficient restoring force to act on the working plunger 12 during the working process of the compressed gas motor 1. Preferably, the steel spring 22 is sufficiently long such that it pushes the working plunger 12 onto the control plunger 14 thus pressing the control plunger 14 against the rear plate 6, in the starting state shown in FIG. 1. Namely, this ensures that the gas inlet opening 24 remains open when no compressed gas is being fed into the compressed gas motor 1 and it ensures that the compressed gas motor 1 is always transitioned into the starting state.

The gas inlet opening 24 is a through-going opening in the side wall of the hollow body 2. Aside from the gas inlet opening 24, a gas outlet opening 26 and a liquid supply opening 28 are provided in the side wall of the hollow body 2. In the starting state of the compressed gas motor 1 as shown in FIG. 1, the gas outlet opening 26 is closed and/or covered by the control plunger 14. The working medium (the compressed gas) is blown off from the internal space through the gas outlet opening 26 when the gas outlet opening 26 is exposed through a motion of the control plunger 14. In contrast, the liquid supply opening 28 is situated between the conveying plunger 12 and the cover plate 4 and is provided for filling the internal space in this region with a liquid, which is then pressed out through the ejection opening 8 upon a motion of the working plunger 12 and thus generates a spray jet (not shown).

FIG. 1 shows the starting state of the compressed gas motor 1. A compressed gas is fed into the internal space between the working plunger 12 and the control plunger 14 via the gas inlet opening 24. The increasing pressure in this intervening space accelerates the working plunger 12 in the direction of the cover plate 4, i.e. towards the front side of the compressed gas motor 1. The control plunger 14 stays in place in this context. This situation is shown in FIG. 2. If a liquid is present in the internal space between the working plunger 12 and the cover plate 4, this liquid is expelled through the ejection opening 8 through the motion of the working plunger 12. Liquid is being ejected for as long as the working plunger 12 moves in the direction of the cover plate 4.

During the motion of the working plunger 12, the working plunger 12 compresses the spring element 22. Once the working plunger 12 has moved away from the control plunger 14 far enough such that the distance is equal to the length of the catch pin 18 minus the thickness of the cover of the control plunger 14, the catch 20 hits against the rear side of the cover of the control plunger 14 and knocks the same forward. The control plunger 14 is then pulled along by the working plunger by means of the catch element 18, 20. As a result, the control plunger 14 moves away from the rear plate 6. The gas inlet opening 24 becomes covered and/or closed through the motion of the control plunger 14. This situation is shown in FIG. 3.

During the whole time, the content of the internal space between the working plunger 12 and the cover plate 4 is expelled through the ejection opening. Due to the inertia of the working plunger 12 and/or control plunger 14 and/or the over-pressure that is still present in the internal space between the working plunger 12 and the rear plate 6, the control plunger 14 is moved forward further and the gas outlet opening 26 is being opened. This situation is shown in FIG. 4.

Subsequently, the gas flows out from the internal space through the gas outlet opening 26 and is released into the surroundings. The spring element 22 accelerates the working plunger 12 in the direction of the rear plate 6. Since the volume between the working plunger 12 and the cover plate 4 increases, an under-pressure is generated in this space and the lip valve 10 closes. More liquid flows in through the liquid supply opening 28 into the enlarging intervening space between the working plunger 12 and the cover plate 4. Preferably, a non-return valve (not shown) is arranged on the liquid supply opening 28 and prevents the liquid from exiting from the intervening space between the working plunger 12 and the cover plate 4 into the liquid supply.

Due to its motion, the working plunger 12 detaches the catch 20 from the rear side of the cover of the control plunger 14. This situation is shown in FIG. 5.

The working plunger 12 finally hits against the control plunger 14 and/or the spacer 16 of the control plunger 14. This also moves the control plunger 14 in the direction of the rear plate 6 again and the gas outlet opening 26 begins to close. This condition is shown in FIG. 6.

Finally, the gas inlet opening 24 also opens again and the spring element 22 has transitioned the working plunger 12 and the control plunger 14 back into the starting state shown in FIG. 1. During this period, the intervening space between the working plunger 12 and the cover plate 4 has become re-filled with liquid or a liquid-gas mixture from the liquid supply opening 28. The cycle starts up again from the beginning.

The control plunger 14 and its motion effect an automatic valve control of the gas inlet opening 24 and gas outlet opening 26 such that the control plunger 14 can be considered to be a valve element.

Due to the action of the compressed gas during the beginning of the motion of the working plunger 12 in the direction of the restoring element 22, the air present in the hollow space is pressed out through the ejection opening 8 and the outlet valve 10. A non-return valve (not shown) arranged on the liquid supply opening 28 prevents the air from exiting into the liquid reservoir. Once the gas outlet opening 26 is opened and the working plunger 12 moves in the direction of the starting position, the force of the restoring element 22 generates an under-pressure in the hollow space. In this context, the non-return valve opens and the liquid flows into the hollow space. Once the gas outlet opening 26 is closed, the effect of the compressed gas moves the working plunger 12 in the direction of the restoring element 22. In this context, the liquid is pressed out through the outlet valve 10, whereby the non-return valve is closed at this time. Once the gas outlet opening 8 is opened, the restoring element 22 presses the working plunger 12 back into its starting position, whereby the under-pressure opens the non-return valve and liquid is aspirated and/or pushed into the internal space. Then, the pumping process of the liquid proceeds in the manner described for as long as compressed gas is being applied to the gas inlet opening 24 and liquid is present in the liquid reservoir.

FIG. 7 shows a schematic cross-sectional view of an alternative inventive compressed gas motor 1 having a tension spring 22 as restoring element 22 that is connected to a rear plate 6 by supports 30 and is connected on the opposite side to a catch 20 by means of supports 32. Moreover, the compressed gas motor 1 comprises a non-return valve 34. Moreover, the shape of the catch 20 and the engagement in the control plunger 14 are designed to be conical. The feed-through through the control plunger 14 through which the catch pin 18 extends presently also serves for gas exchange between the intervening space between the working plunger 12 and the control plunger 14 and the intervening space between the control plunger 14 and the rear plate 6. There are no other feed-throughs in this embodiment. In all other respects, the structure and functional principle are identical to those shown in FIGS. 1 to 6 and described above.

The tension spring 22 pulls the working plunger 12 in the direction of the rear plate 6 to the extent that the control plunger 14 touches against the rear plate 6 and the working plunger 12 touches against the control plunger 14. Preferably, the tension spring 22 then still exerts a force on the working plunger 12 that pulls the working plunger 12 in the direction of the rear plate 6. The operating principle of the compressed gas motor 1 according to FIG. 7 is the same as the functional principle of the compressed gas motor 1 according to FIGS. 1 to 6 except that the spring 22 according to FIG. 7 is being tensioned by extension, i.e. it is a tension spring, whereas the spring 22 according to FIGS. 1 to 6 takes up potential energy through compression.

The non-return valve 34, which, in the same design, can also be arranged on the liquid supply openings 28 of other compressed gas motors, like the one according to FIGS. 1 to 6, consists of a ball that is pressed onto a ball seat by means of a steel spring. This enables a flow through the liquid supply opening 28 into the internal space of the hollow body 2, whereas a flow out of the internal space is blocked through the non-return valve 34. Accordingly, if the content of the compressed gas motor 1 between the working plunger 12 and the cover plate 4 is sprayed out through the ejection opening 8 by means of a motion of the working plunger 12 towards the cover plate 4, the non-return valve 34 prevents the content from advancing into the supply line.

The compressed gas-driven liquid pump 1 works such that a hollow space is present on the front side of the working plunger 12 at which the restoring element 22 engages. Said hollow space is connected in liquid-permeable manner to the non-return valve 34 that enables an inflow of liquid from a liquid reservoir into the hollow space and prevents the liquid from flowing in reverse from the hollow space in the direction of the liquid reservoir. Moreover, an outlet valve 10 is connected to the hollow space in liquid-permeable manner. Said outlet valve enables the liquid to flow out of the hollow space and prevents the liquid from flowing back into the hollow space. Said outlet valve 10 can be a lip valve 10 in the simplest case. Alternatively, the restoring element 22 can just as well be situated in the hollow space (see FIGS. 1 to 6). The basic functional principle of the compressed gas motor 1 is as described above.

FIG. 8 shows a schematic cross-sectional view of a third alternative inventive compressed gas motor 1 having a rod 40 for forming a push-type compressed gas motor 1. The structure of the compressed gas motor 1 is the same as those of the afore-described compressed gas motors according to FIGS. 1 to 6 and 7. The compressed gas motor 1 comprises a hollow space 2 that has a cylindrical internal space. The internal space is covered by a cover plate 4 on the front side and is closed on the rear side by a rear plate 6 in gas- and pressure-tight manner. A working plunger 12 is arranged in the internal space such as to be mobile in linear direction along the cylinder axis of the internal space. A control plunger 14 is arranged between the working plunger 12 and the rear plate 6 such as to be mobile in linear direction along the cylinder axis of the internal space. Working plunger 12 and control plunger 14 comprise a cylindrical outer jacket that corresponds to the inner cylinder jacket of the internal space.

The working plunger 12 is sealed with respect to the internal wall of the internal space through two sealing rings 15. A cylindrical sleeve is arranged as spacer 16 on the front side of the control plunger 14. Passages 17 are arranged in the walls of the spacer and enable gas exchange between the front side and the rear side of the control plunger 14.

A catch element consisting of a catch pin 18 and a catch 20 is fastened to the rear side of the working plunger 12. The catch pin 18 extends through a feed-through in the control plunger 14 such that the catch 20 can engage on the rear side of the control plunger 14 in order to pull the control plunger 12 along at a distance to the working plunger 12 when the working plunger 12 moves in the direction of the front side, i.e. in the direction of the cover plate 4.

A spring element 22 is arranged in the internal space between the working plunger 12 and the cover plate 4 and about the rod 40 that pushes the working plunger 12 in the direction of the rear plate 6 and, in FIG. 8, onto the control plunger 14. FIG. 8 also shows the starting position of the compressed gas motor 1.

Two through-going openings 24, 26 are provided in the side wall of the hollow body 2. A compressed gas is fed into the compressed gas motor 1 at the gas inlet opening 24.

During the working cycle of the compressed gas motor 1, the compressed gas is expelled again through the gas outlet opening 26. The working cycle proceeds as described through the first exemplary embodiment according to FIGS. 1 to 6.

Instead of expelling a liquid periodically, as with the lavage system according to FIGS. 1 to 6, the compressed gas motor 1 attains periodical pushing by the rod 40. The rod 40 is guided, by means of a guide sleeve 41, through an opening in the cover plate 4 out of the internal space. Alternatively, instead of the rod 40 being rigidly connected to the working plunger 12, a plunger rod 40 can be connected to the working plunger 12 by means of a joint (not shown) that is guided through the cover plate 4. The plunger rod 40 can be used to translate the periodical linear motion into a rotary motion by means of a crankshaft (not shown) that is fastened in the front to the plunger rod 40 by means of a joint.

The compressed gas motor 1 according to FIG. 8 can also be used in a lavage system. For this purpose, the tip of the rod 40 is used to hit against a membrane (not shown). The periodical impact on the membrane can be used for periodical ejection of a liquid. Likewise, the periodical motion of the rod 40 can be used as a drive for a shaking mechanism or a rapping motor.

Alternatively, the compressed gas motor 1, if provided with a plunger rod 40, can be used to drive a cutting disc or a toy or a pump.

FIG. 9 shows a schematic cross-sectional view of a fourth alternative inventive compressed gas motor 1 having a string 42 or a cable 42 as catch element 42. In this version of the compressed gas motor 1, the spacer 16 is arranged on the rear side of the working plunger 12. Presently, the working plunger 12 is sealed with respect to the internal walls of the cylindrical internal space in the hollow body 2 just by an O-ring 15.

In the position of the compressed gas motor 1 shown in FIG. 9, the cables 42 are just tensioned. The working plunger 12 pulls the control plunger 14 along in trailing manner in the direction of the cover plate 4 in order to close the gas inlet opening 24 and then to open the gas outlet opening 26. The cables 42 are fastened to struts 43 on the front side of the control plunger 14, for example are tied around the struts 43 by means of a loop.

When the working plunger 12 with the spring element 22 is pushed in the direction of the rear plate 6, the cables 42 are loosely attached in the internal space between the working plunger 12 and the control plunger 14. Only when the control plunger 14 is pulled along by the working plunger 12, the cables 42 or strings 42 are tensioned to be taut, as shown in FIG. 9.

FIG. 10 shows a schematic cross-sectional view of a fifth alternative inventive compressed gas motor 1, in which the catch element 18, 20 is fastened to the control plunger 14. In this embodiment, the catch element 18, 20 runs in a hollow space in the working plunger 12. Since the catch pin 18 is longer than the hollow space in the working plunger 12 is deep, it also assumes the task of a spacer that ensures that the working plunger 12 is definitely situated at a distance from the control plunger 14 such that the compressed gas, in the starting position shown in FIG. 10, can flow through the gas inlet opening 24 between the working plunger 12 and the control plunger 14 in order to accelerate the working plunger 12 in the direction of the cover plate 4.

The passages 17 in the control plunger 14 are necessary to allow the compressed gas in the compressed gas motor 1 to flow to the rear side of the control plunger 14 in order to finally flow out through the gas outlet opening 26 when said opening is opened by the control plunger 14.

FIG. 11 shows a schematic cross-sectional view of a sixth alternative inventive compressed gas motor 1 having a smaller working space between a working plunger 12 and a control plunger 14 as compared to the preceding embodiments. The working space is reduced by reducing the internal diameter of the hollow body 2 and of the plungers 12, 14. For this purpose, the working plunger 12 must comprise two different cross-sections (or two different diameters if this concerns a cylindrical structure with a circular base surface) that match the cross-sections of the two cylindrical internal spaces. As a result of having the smaller working space, less compressed gas is consumed per motion cycle of the compressed gas motor 1. As a result, the motion is somewhat less forceful. But it is still sufficient for the use as a spray pump for a lavage system.

The cylindrical internal space according to the invention, inside which the working plunger 12 moves, presently is the one having the smaller cross-section since this is where the motion of the working plunger 12 is generated by the compressed gas. The working cycle and the pumping process and/or spraying process are no different from those of the working cycle described with reference to FIGS. 1 to 6. FIG. 11 again shows the starting state of the compressed gas motor 1 and/or working plunger 12 and control plunger 14 therein. As before, a non-return valve (not shown) is arranged on the liquid supply opening 28 and is used to prevent any ingress of liquid from the pumping space (front part of the space formed by the hollow body) into the liquid supply.

FIG. 12 shows a schematic cross-sectional view of a lavage system 50 according to the invention that can be held in one hand and has a compressed gas motor 1 according to the invention. Except for the arrangement of the gas inlet opening 24, gas outlet opening 26, and liquid supply opening 28 with respect to each other, the compressed gas motor 1 is structured alike the compressed gas motor 1 according to FIGS. 1 to 6 such that reference shall be made to said exemplary embodiment with respect to the structure.

The lavage system 50 comprises a housing 52 made of plastic material, in which the compressed gas motor 1 is arranged. A compressed gas cartridge 54 is arranged in the housing 52 in an opening on the rear side (on the right in FIG. 12) and contains a compressed gas or a liquefied compressed gas such as, for example, CO₂, which drives the compressed gas motor 1. A handle 55 is arranged on the rear side of the compressed gas cartridge 54 and can be used to screw or push the compressed gas cartridge 54 into the lavage system. The gas cartridge 54 is a CO₂ cartridge made of metal and is pushed by means of the handle 55 onto a mandrel (not shown) of a connecting unit 56 such that the mandrel opens the compressed gas cartridge 54 and the compressed gas flows from the compressed gas cartridge 54 into the connecting unit 56.

The liquefied gas from the compressed gas cartridge 54 then flows from the connecting unit 56 through a compressed gas line into an evaporation container 58, in which liquid components of the gas can evaporate. In turn, the evaporation container 58 is connected to a pressure-reducing valve 60 by means of a compressed gas line. The pressure-reducing valve 60 can be operated by means of an adjusting screw 62 situated on the upper side of the lavage system. The pressure reducing valve 60 can be used to adjust the gas pressure as desired to operate the compressed gas motor 1.

The compressed gas is passed by the pressure-reducing valve 60 via a gas feed line 68 to a control valve 64. The control valve 64 can be operated manually by means of a trigger 66 of the type of a pistol using the same hand that holds the lavage system. The gas feed line 68 continues downstream from the manually operable control valve 64 and is connected to the gas inlet opening 24 of the compressed gas motor 1.

The liquid supply opening 28 is connected to a rinsing liquid supply line 70, in which a non-return valve 34 is arranged. An opening 72 is provided on the top of the housing 52 through which the compressed gas from the compressed gas motor 1 escaping through the gas outlet opening 26 can exit. The opening 72 can be covered by a grid and/or a filter.

The evaporation container 58 is arranged in a handle part 74 of the type of a pistol handle that is formed by the plastic housing 52. A tube 76 having a dispensing opening 78 and a funnel 79 is arranged on the front side of the lavage system and allows the liquid puffs from the compressed gas motor 1 to exit from the lavage system.

In lavage systems of this type, it is preferred to provide an aspiration facility (not shown) by means of which excess liquid and parts removed along with the liquid can be aspirated and discharged. The lavage system is ready for use as soon as a compressed gas cartridge 54 is inserted into it. When the control valve 64 is operated by the trigger, the gas supply line 68 becomes through-going and the compressed gas is fed from the pressure-reducer 60 into the compressed gas motor 1. The compressed gas puts the working plunger 12 and subsequently the control plunger 14 in motion and the compressed gas motor 1 works in the manner described above. Concurrently, the liquid conveyed from an external liquid reservoir (not shown) through the liquid supply line 70 flows periodically into the intervening space between the working plunger 12 and the cover plate 6 of the compressed gas motor 1, in which the spring 22 as restoring element 22 is situated as well.

The liquid is expelled from the compressed gas motor 1 through the tube 76 and the dispensing opening 78 by periodical impacts for as long as the compressed gas flows through the gas inlet opening 24 into and through the compressed gas motor 1. The process is terminated when the compressed gas cartridge 54 is empty or the control valve 64 is no longer operated and the compressed gas supply line 68 is thus interrupted. The control valve 64 is restored, for example, by means of an elastic spring. The working plunger 12 and the control plunger 14 are then positioned in the starting position shown (see FIG. 1 as well) by the spring element 22 in the compressed gas motor 1 and the lavage system is immediately ready for use again.

Instead of just one compressed gas motor 1, a lavage system 50 can just as well comprise two or more compressed gas motors 1, whose compressed gas connectors are arranged parallel to each other. This effects a reinforcement of the spray jet thus generated or attains a higher pulse rate of doubled frequency. A lavage system of this type is shown in a schematic top view in FIG. 13. The lavage system according to FIG. 13 comprises two compressed gas motors 1 which are arranged in parallel with respect to two compressed gas supply lines 68. The compressed gas supply lines 68 are supplied with compressed gas by means of a pressure reducer 60. For this purpose, the pressure reducer 60 is connected to a compressed gas cartridge 54 by means of a connecting unit 56 and an evaporation container 58.

The two compressed gas motors 1 each exit via a tube 76 into a funnel 79 from which the liquid flows are dispensed. An aspiration facility 80 is provided on the funnel 79 and is used to aspirate the liquid and residues rinsed out together with the liquid.

The features of the invention disclosed in the preceding description and in the claims, figures, and exemplary embodiments, can be essential for the implementation of the various embodiments of the invention both alone and in any combination.

### LIST OF REFERENCE NUMBERS

1 Compressed gas motor
2 Hollow body
4 Cover plate
6 Rear plate
8 Ejection opening
10 Lip valve
12 Working plunger
14 Control plunger
15 Seal/O-ring
16 Spacer
17 Passage
18 Catch pin
20 Catch
22 Spring element
24 Gas inlet opening
26 Gas outlet opening
28 Liquid supply opening
30, 32 Support
34 Non-return valve
40 Rod/plunger rod
41 Guide sleeve
42 Cable/string
43 Strut
50 Lavage system
52 Housing
54 Gas pressure cartridge
55 Handle for gas pressure cartridge
56 Connecting unit for gas cartridge
58 Evaporation container
60 Pressure-reducing valve
62 Adjusting screw
64 Control valve
66 Trigger
68 Gas supply line
70 Rinsing liquid supply line
72 Opening
74 Handle part
76 Tube
78 Dispensing opening
80 Aspiration opening

1. A compressed gas motor comprising a working plunger, an internal space that is closed on a rear side and has the working plunger arranged such that the working plunger is mobile in linear direction, a restoring element that exerts a force on the working plunger, at least part of the time, in the direction of the rear side, a gas inlet opening for supplying a compressed gas into the internal space and a gas outlet opening for discharging the gas from the internal space, the compressed gas motor comprising:
a control plunger arranged in the internal space between the working plunger and the closed rear side such as to be mobile in linear direction, whereby the control plunger, in a first position, covers the gas outlet opening and does not cover the gas inlet opening and, in a second position, covers the gas inlet opening and does not cover the gas outlet opening, the control plunger is supported to be mobile with respect to the working plunger, and
a catch element that is arranged on the working plunger and/or control plunger and pulls the control plunger in the direction of the working plunger, at a first distance, when the working plunger moves away from the control plunger.

2. The compressed gas motor according to claim 1, wherein the control plunger, in a third position between the first position and the second position, covers both the gas inlet opening and the gas outlet opening.

3. The compressed gas motor according to claim 1, wherein at least one spacer is arranged on the side of the control plunger facing the working plunger and/or at least one spacer is arranged on the side of the working plunger facing the control plunger, such that an intervening space arises between the working plunger and the control plunger when the working plunger and control plunger touch against each other, and the gas inlet opening, in the first position, exits into said intervening space, wherein the at least one spacer adjusts a second distance between the working plunger and the control plunger when working plunger and control plunger touch against each other, wherein the second distance is smaller than the first distance that is being adjusted by the catch element.

4. The compressed gas motor according to claim 1, wherein the restoring element is an elastic spring that is arranged in the internal space between the working plunger and a front end of the internal space, wherein the front end of the internal space is arranged opposite from the rear side of the internal space.

5. The compressed gas motor according to claim 1, wherein the control plunger, in the first position, is pushed or pulled over the working plunger against the rear side by the restoring element, whereby the gas inlet opening exits into an opening or into the intervening space between the working plunger and the control plunger.

6. The compressed gas motor according to claim 1, wherein at least one gas-permeable passage is arranged in the control plunger and connects the front side of the control plunger facing the working plunger to the rear side of the control plunger facing the rear side of the internal space.

7. The compressed gas motor according to claim 1, the working plunger touches against the internal wall of the internal space by its entire circumference, in a gas-tight and pressure-tight manner, by means of a sealing element.

8. The compressed gas motor according to claim 1, wherein a plunger rod is attached to the side of the working plunger that faces away from the control plunger and projects from the internal space through the front side of the internal space.

9. The compressed gas motor according to claim 8, wherein the plunger rod is guided through a gas-tight feed-through through the front side of the internal space out of the internal space such that the internal space is closable in a gas-tight and pressure-tight manner between the front side and the working plunger to form a gas-operated spring as restoring element.

10. The compressed gas motor according to claim 1, wherein an ejection opening is provided in the front side of the internal space opposite from the rear side, and a liquid supply opening is arranged in at least one of the front side and the lateral wall of the internal space and the opening is not covered by the working plunger, at least for part of the time, and, in the non-covered state, is arranged between the working plunger and the front side of the internal space.

11. The compressed gas motor according to claim 10, wherein the ejection opening is connected to the surroundings by a valve element, wherein the valve element is opened in the presence of sufficient over-pressure as compared to the ambient pressure and is closed otherwise, and further wherein a tube or a hose with a non-return valve is connected on the liquid feed opening and opens in the presence of an under-pressure in the internal space between the working plunger and the front side of the internal space and thus enables liquid to be supplied into the internal space.

12. The compressed gas motor according to claim 1, wherein the catch element is a string, a cable, a thread, a chain or an elastic spring that is attached to the working plunger and to the control plunger or the catch element comprises a rod, a string, a cable, a thread, a chain or an elastic spring that is attached to the working plunger or to the control plunger and has a catch attached to the catch element that engages a projection in the working plunger or in the control plunger, wherein the catch element is provided by a rod that is attached to the working plunger and extends through a feed-through in the control plunger and has a catch attached to it that does not fit through the feed-through in the control plunger and engages the control plunger on the rear side thereof in order to pull the control plunger along, when the working plunger is remote with respect to the control plunger and moves in the direction away from the control plunger.

13. The compressed gas motor according to claim 1, wherein the internal space, at least regions thereof, is cylindrical or is cylindrical in the region of a working space of the working plunger or in the entire swept volume of the working plunger and control plunger.

14. The compressed gas motor according to claim 1, wherein the working plunger comprises two differently-sized cross-sectional surfaces perpendicular to the motion direction of the working plunger, wherein the internal space comprises matching internal walls with different cross-sectional surfaces and the cross-sectional surface on the side of the working plunger facing the rear side is larger than the cross-sectional surface of the opposite front side of the working plunger.

15. A lavage system comprising at least one compressed gas motor according to claim 1, wherein the at least one compressed gas motor is usable to generate a periodical spray puff of a liquid.

16. The lavage system according to claim 15, wherein the lavage system comprises a connector for a compressed gas cartridge, a pressure reducer, an evaporation space for evaporation of liquid residual liquid gas, and a manually actuated control valve, wherein the evaporation space is connected to the connector by means of a compressed gas line, the pressure reducer is connected to the evaporation space by means of a compressed gas line, the control valve is connected to the pressure reducer by means of a compressed gas line, and the gas inlet opening of the compressed gas motor is connected to the control valve by means of a compressed gas line, and further wherein a liquid supply opening of the compressed gas motor is connected to a liquid line.

17. The compressed gas motor according to claim 1, wherein the compressed gas motor is one selected from the group consisting of a motor for a lavage system, a rapping motor, a motor for a toy, a vibration motor, a drive for a dosing facility, a shaker motor or a pump.

18. A method for generating a periodical motion with a compressed gas, the method comprising the steps of:
providing the compressed gas motor according to claim 1, wherein the working plunger and the control plunger, in a starting state, are situated in the internal space such as to be at the first distance from each other, wherein the control plunger closes the gas outlet opening and the gas inlet opening between the working plunger and the control plunger is open;
supplying the compressed gas into the internal space between the working plunger and the control plunger;
accelerating and moving, via the pressure of the compressed gas, the working plunger in the direction of a front side of the internal space away from the control plunger, wherein the distance between working plunger and control plunger increases;
pulling the control plunger along by the working plunger by means of the catch element, as soon as the second distance is reached;
closing, via the control plunger, the gas inlet opening due to the motion of the control plunger;
opening, via the control plunger, the gas outlet opening due to the motion of the control plunger;
accelerating, via the restoring element, the working plunger in the direction of the rear side of the internal space;
flowing the compressed gas between the working plunger and the control plunger through at least one gas-permeable opening through the control plunger into the space between the rear side of the control plunger and the rear wall of the internal space;
releasing the compressed gas in the space between the rear side of the control plunger and the rear wall of the internal space into the surroundings through the open gas outlet opening;
moving the working plunger against the control plunger and in the direction of the rear side of the internal space;
closing the gas outlet opening again by the reverse motion of the control plunger; and
opening, via the reverse motion of the control plunger, the gas inlet opening such that compressed gas is supplied into the internal space between working plunger and control plunger.

19. The method according to claim 18, wherein the steps repeat upon compressed gas being supplied again.

20. The method according to claim 18, wherein the first distance between working plunger and control plunger is adjusted by means of at least one spacer and the second distance between working plunger and control plunger is adjusted by a catch element.

21. A method for generating a spray puff, the method comprises the steps according to the method of claim 18 and further comprising the steps of:
ejecting, upon a motion of the working plunger away from the rear side of the internal space, a liquid or a liquid-gas mixture from the space between the working plunger and the front side of the internal space through an ejection opening on the front side of the internal space; and
pushing or pulling, upon a motion of the working plunger towards the rear side of the internal space, a liquid or a liquid-gas mixture through a liquid supply opening into the space between the working plunger and the front side of the internal space.

22. The method according to claim 21, wherein, upon the motion of the working plunger towards the front side of the internal space, the pressure in the space between the working plunger and the front side of the internal space opens and/or keeps open a valve at the ejection opening and closes and/or keeps closed a non-return valve connected to the liquid supply opening, and further wherein, upon the motion of the working plunger towards the rear side of the internal space, the lesser pressure in the space between the working plunger and the front side of the internal space closes and/or keeps closed the valve on the ejection opening and opens and/or keeps open the non-return valve connected to the liquid supply opening.
